(19) United States
(12) Patent Application Publication
(10) Pub. No.: US 2007/0047848 A1
(43) Pub. Date: Mar. 1, 2007

(54) CONVERTIBLE TOTE BAG AND SUNTAN SHIELD

(76) Inventor: Dianne Kenney, Grande Prairie (CA)

Correspondence Address: EDWARD YOO C/O BENNETT JONES, 1000 ATCO CENTRE, 10035 - 105 STREET, EDMONTON, ALBERTA, AB, T5J3T2, CA

(21) Appl. No.: 11/164,495

(22) Filed: Nov. 25, 2005

(30) Foreign Application Priority Data
Mar. 11, 2005 (CA) 2,500,440

(51) Int. Cl. B65D 30/00; B65D 33/28; A61F 9/00

(52) U.S. Cl. 383/4; 383/72; 2/12

(57) ABSTRACT

A convertible tote bag may be used to carry personal items and to cover a user's head during suntanning. The bag may include a shield which may be deployed to cover a user's face during suntanning.

### CROSS REFERENCE TO RELATED APPLICATIONS

This application claims the priority benefit of Canadian Patent Application 2,500,440 filed on Mar. 11, 2005 entitled “Convertible Tote Bag and Suntan Shield”, the contents of which are incorporated herein by reference.

### FIELD OF INVENTION

The present invention relates to a convertible tote bag and suntan shield.

### BACKGROUND OF THE INVENTION

Tanning salons have become increasingly popular. There are a variety of reasons why people use tanning beds. Some tan before sunny destination trips or before summer for a base tan to avoid burning, while others tan before special events like weddings, graduation ceremonies, or parties. In general, a tan is seen to be attractive and desirable. However, exposure to UV light can damage or alter the colour of hair. This is particularly so if the hair has been recently coloured or permed. Therefore, many tanning enthusiasts will cover their hair with a towel or other means. Also, some people do not wish to have their faces tan as darkly as other parts of their body and will cover their face while tanning.

It would be desirable to have a bag which may be useful as a tote bag, and to cover parts of a person's body while tanning.

### SUMMARY OF THE INVENTION

The present invention relates to a convertible bag for use as a tote bag and as a tanning shield. The bag defines an open end and an interior volume and comprises:

(a) means for closing the open end;

(b) a face shield associated with the open end, wherein the shield may be inserted within the interior volume and extracted to a position outside of the bag;

(c) wherein the bag comprises a material which substantially blocks ultraviolet radiation;

(d) and wherein the bag and open end are sized to fit over a human head.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is a schematic representation of one embodiment of the present invention.

FIG. 2 shows the present invention in use as a tote bag.

FIG. 3 shows the present invention in use as a tanning cap with the face shield flipped up.

FIG. 4 shows the present invention in use as a tanning cap with the face shield flipped down.

### DETAILED DESCRIPTION OF THE INVENTION

The present invention provides for a convertible tote bag which may also be used as a tanning cap and face shield. When describing the present invention, all terms not defined herein have their common art-recognized meanings.

As the present invention is intended to be used to block, or partially block, ultraviolet radiation (UVR), it is preferably constructed from material or fabric which is relatively opaque to UVR. It is well known that:

1. less UVR passes through tightly woven or knitted fabrics;
2. darker colours usually block more UVR; and
3. heavier weight fabrics usually block more UVR than light fabrics of the same type.

A fabric's ability to block UVR is gauged by an ultraviolet protection factor (UPF) rating which is a standard adopted in Australia and New Zealand.

UPF Ratings and Protection Categories

| UPF Rating | Protection Category | % UVR Blocked |
|---|---|---|
| 15-24 | Good | 93.3-95.9 |
| 25-39 | Very Good | 96.0-97.4 |
| 40 and over | Excellent | 97.5 or more |

It is preferable that the fabric used in the present invention have a UPF rating of about 15 or greater, however, any fabric which blocks a substantial portion of UVR (at least 50%), may be suitable.

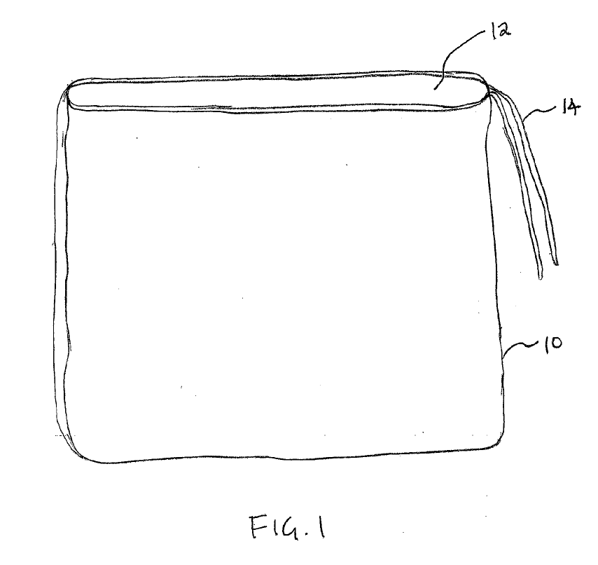

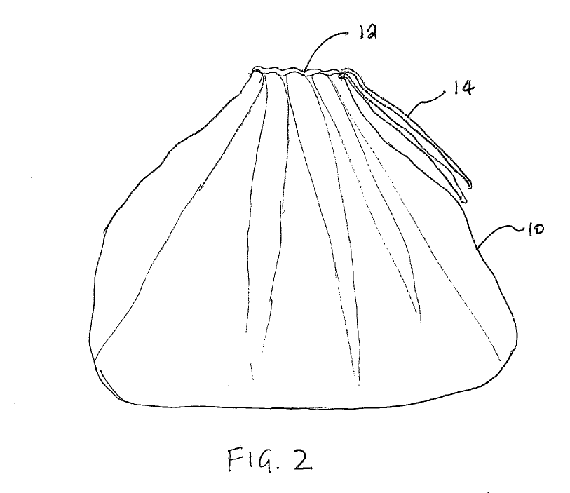

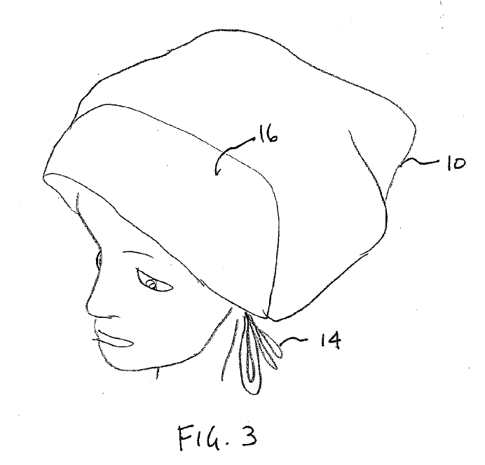

The fabric is sewn or otherwise fashioned into a bag (10) shape, as shown in FIG. 1, with one open end (12). The bag (10) and opening (12) are sized to fit human heads. The open end preferably has a draw string (14) associated with the periphery of the opening to close the bag (10) as may be seen in FIG. 2, or to tighten the bag (10) around a user's head, as may be seen in FIG. 3. The draw strings (14) may provide a convenient handle to carry the bag when closed. Such draw strings are well known in the art and need no further description. Alternatively, or additionally, the opening may be elasticized.

Alternative means of closing the open end (12) may be provided, including such well known means as zippers, snaps, hook and loop fasteners and the like. However, many such closure means are not preferred as they may be uncomfortable when the bag is worn over the user's head.

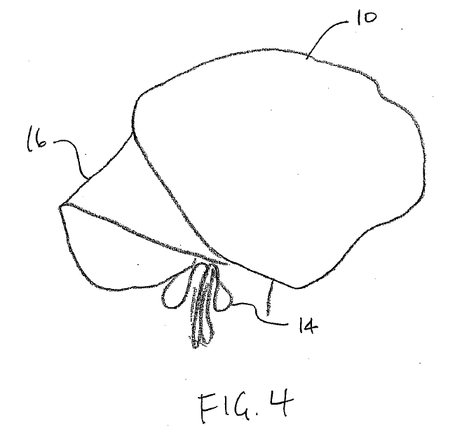

The bag may further comprise a face shield (16) formed from a flap of material attached to the open end. When used as a tote bag, the shield (16) may be tucked inside the bag (10, as seen in FIG. 2. When in use on a user's head, the shield (16) may be tucked within the bag, or maybe flipped upwards away from the user's face, as may be seen in FIG. 3, or may be lowered to shield the user's face, as may be seen in FIG. 4.

As will be apparent to those skilled in the art, various modifications, adaptations and variations of the foregoing specific disclosure can be made without departing from the scope of the invention claimed herein. The various features and elements of the described invention may be combined in a manner different from the combinations described or claimed herein, without departing from the scope of the invention.

What is claimed is:

1. A convertible bag having an open end and defining an interior volume, comprising:
(a) means for closing the open end;
(b) a face shield attached to the bag and associated with the open end, wherein the face shield may be inserted within the interior volume and extracted to a position outside of the bag;
(c) wherein the bag comprises a material which substantially blocks UVR;
(d) and wherein the bag and open end are sized to fit over a human head.

2. The convertible bag of claim 1 wherein the means for closing the open end comprises a draw string.

3. The convertible bag of claim 2 wherein the draw string comprises a handle when the draw string closes the open end.
